(19) United States
(12) Patent Application Publication
(10) Pub. No.: US 2006/0173393 A1
(43) Pub. Date: Aug. 3, 2006

(54) Inflatable splint for stabilisation of the ankle

(75) Inventor: Pascal Sailhen, Lyon (FR)

Correspondence Address: OLIFF & BERRIDGE, PLC, P.O. BOX 19928, ALEXANDRIA, VA, 22320, US

(73) Assignee: ORHMIHL-DANET, VILLEURBANNE (FR)

(21) Appl. No.: 10/541,554

(22) PCT Filed: Feb. 18, 2004
(86) PCT No.: PCT/FR04/00368

§ 371 Date: Aug. 18, 2005

(30) Foreign Application Priority Data
Feb. 25, 2003 (FR) 03/02291

(51) Int. Cl. A61F 5/00

(52) U.S. Cl. 602/23; 602/13; 602/27

(57) ABSTRACT

The brace (1) comprises two lateral shells (2, 3) with which are associated, respectively, two bags (12, 13) which are inflatable with air and which bear on the right and left sides of the ankle, while straps (4, 5) connect the two shells (2, 3) and permit tightening of the brace (1) around the ankle. The two bags (12, 13), respectively right and left, are equipped with means of inflation (17, 18, 19) common to these two bags, with a single inflation valve (19). In their upper half, the two inflatable bags (12, 13) comprise points or lines of connection (21) between their two opposite walls (14, 15), in such a way as to limit the degree of inflation in this zone. This brace (1) can be used to treat ankle sprains.

The present invention generally concerns the technical field of orthotic devices, that is to say devices or accessories whose function is to support, immobilize, maintain, correct or re-educate certain parts of the human body. More particularly, this invention relates to a brace for stabilizing the ankle and, still more especially, to a brace of this kind which can be inflated with air.

As is generally known, braces for stabilizing the ankle are used particularly in cases of ankle sprains, for recovery of posture and for lateral stabilization of the ankle. Among these braces, inflatable braces are already known, that is to say braces which have parts in the form of bags filled with air in such a way as to exert a suitable pressure on certain zones of the ankle of the person wearing the brace, each bag having closure means to permit its inflation with air. Such designs are known, in particular, from European patent No. 0 252 121 (or equivalent document WO 87/03471), or from U.S. Pat. No. 4,628,945.

In these earlier designs, irrespective of the construction details, two lateral shells are provided which bear on either side of the ankle of the patient, each lateral shell being assigned an inflatable bag. Thus, the two inflatable bags, which bear against the right and left sides of the ankle, respectively, are always independent of one another.

The consequence of this is that, when using these known ankle braces, the pressures exerted respectively on the right and left sides of the ankle, as a function of the degree of inflation of the two lateral bags, risk being unequal or asymmetrical, since there is nothing to guarantee here that the inflation, and thus the pressure and tightening, can be suitably balanced between the right and left sides.

Moreover, the independence of the two inflatable bags means that they have to be inflated or deflated separately and successively, which makes the procedures of inflating and deflating the brace relatively long and inconvenient for the user.

Finally, in the existing designs of such braces, the inflatable bags have no special configuration, and they form simple air cushions, each inflated bag thus exerting, on the ankle, a constant pressure along its full extent, whereas it is in fact desirable to be able to vary and adapt this pressure according to the bearing zones. In particular, in the case of an ankle sprain, the pressure exerted by the upper part of the brace, in the area where there is no oedema, should be less, because it is not necessary and could create an undesirable “tourniquet” effect.

The present invention aims to remedy the disadvantages or shortcomings of the existing inflatable ankle braces mentioned above, by providing an improved inflatable ankle brace which permits certain balance of, and a possibility of adapting, the pressures, while at the same time making the inflation and deflation procedures easier.

To this end, the subject of the invention is an inflatable brace for stabilizing the ankle, which comprises two lateral shells with which are associated, respectively, two bags which are inflatable with air and which are intended to bear, respectively, on the right and left sides of an ankle, said bags being provided with means for their inflation, and means of the strap type being provided to connect the two shells and permit tightening of the brace around an ankle, this brace being characterized essentially by the fact that the means of inflation of the two inflatable bags, respectively right and left, are common to these two bags.

In a preferred embodiment of the invention, the common means of inflation comprise two flexible inflation conduits which start, respectively, from the upper parts of the two bags and which converge, in a V-shaped configuration, towards a common inflation valve situated between the two bags. Advantageously, the two flexible conduits and the inflation valve are situated in the front upper part of the brace, which makes the inflation means particularly accessible, in particular to permit inflation or deflation by the user himself, without removing the brace.

By virtue of these means of inflation common to the two bags, it is guaranteed that the inflation pressure of the two bags is identical, hence that the bearing pressures of the inflatable brace against the right and left sides of the ankle are equal and symmetrical, and this guarantees a tightening which is itself symmetrical and balanced.

Moreover, because there is just one inflation valve, and because of its preferred position at the front of the brace, the procedures for inflation and deflation of the two bags are made particularly easy and rapid.

According to a particularly advantageous embodiment of the invention, each of the two inflatable bags has, at least in its upper half, a zone comprising points and/or lines of connection between these two opposite walls, in such a way as to limit, in this zone, the degree of inflation of each bag. In particular, said zone is provided with rectilinear and substantially parallel weld seams connecting the two opposite walls of the bag. Thus, the thickness of the inflation air cushion can be limited in a certain zone of each bag, and in particular in its upper half, by keeping its walls quite close together, so as to locally limit the tightening above the ankle and thereby avoid any undesirable “tourniquet” effect.

According to a complementary characteristic of the invention, each inflatable bag of the brace comprises, in its lower half and on the inner side, a small auxiliary bag which is open in its upper part and is intended for the introduction of removable cushions, in particular of foam or gel. Such accessories, thus placed in the regions of the malleoli, improve patient comfort and/or make it possible to reduce bruising (in cases of ligament rupture), especially if these are cushions of gel which can be cooled in advance.

The invention will be better understood from the following description in which reference is made to the attached diagrammatic drawing which shows, by way of example, an embodiment of this inflatable brace for stabilizing the ankle:

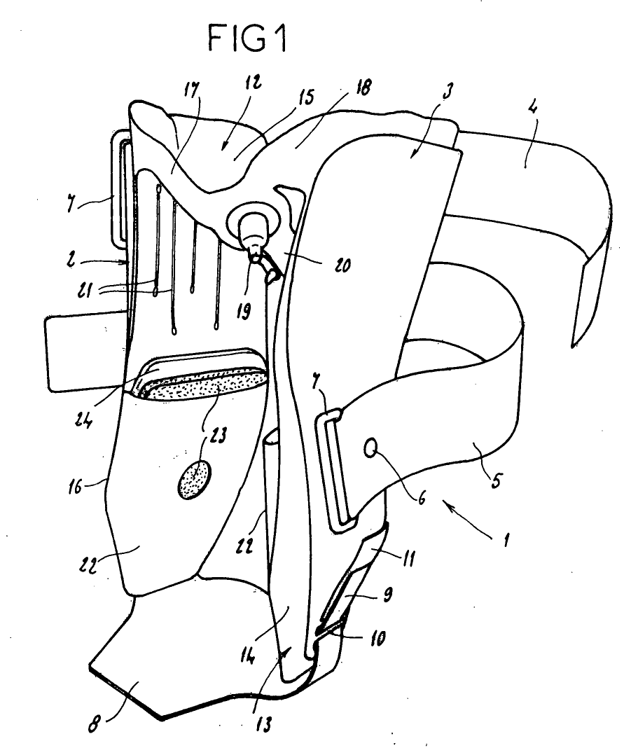

FIG. 1 is a general perspective view of an ankle brace according to the present invention;

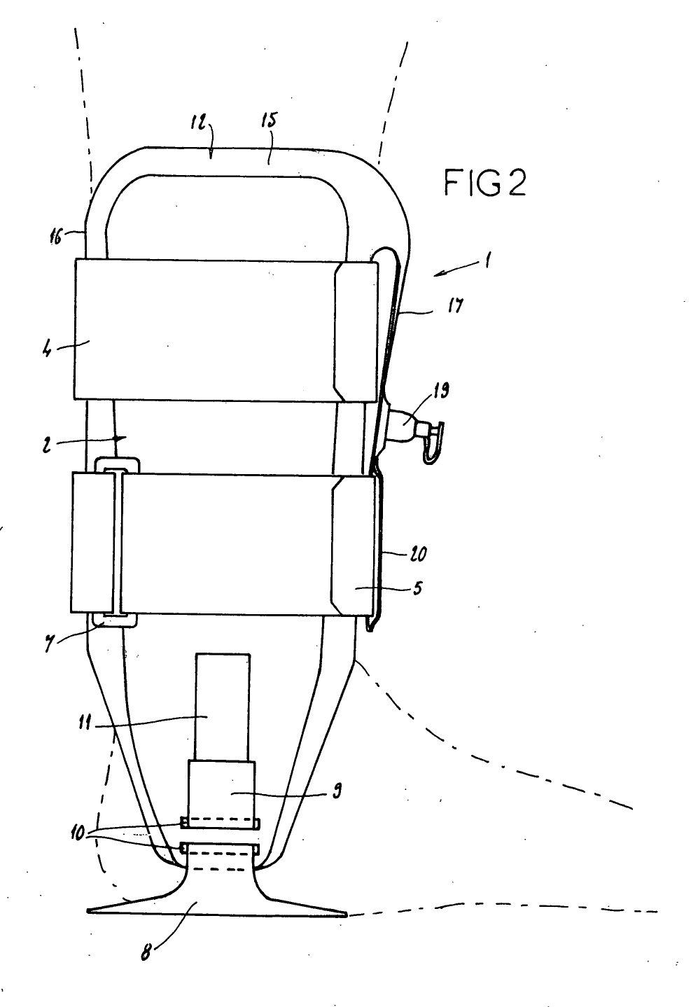

FIG. 2 is a side view of the brace from FIG. 1, positioned on an ankle;

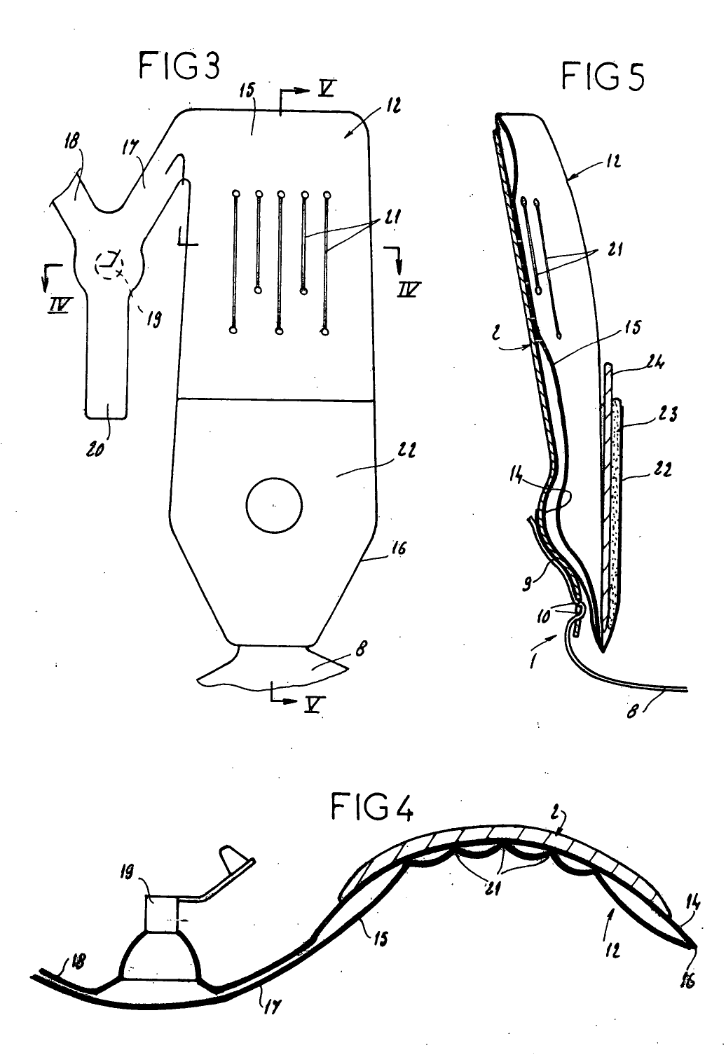

FIG. 3 shows, in a front view, one of the two inflatable bags of this ankle brace, with the means of inflation;

FIG. 4 is a transverse section of the bag and of the inflation means, along line IV-IV in FIG. 3;

FIG. 5 is a vertical section of the same bag, along the line V-V in FIG. 3, indicating the removable cushions of foam and gel that can be introduced there.

FIGS. 1 and 2 show, in its entirety, an ankle brace designated overall by reference number 1.

Of generally symmetrical structure, this brace 1 comprises two semi-rigid lateral shells 2 and 3 which are made of moulded synthetic material and have an anatomical shape allowing them to fit, respectively, to the right and left sides of an ankle, in particular in the region of the malleoli, and also to the lower part of a user's leg.

For the connection of the two shells 2 and 3, two superposed straps 4 and 5 are provided here which are made of self-adhesive tape. Each strap 4 or 5 is fixed on one of the shells 2 or 3 by means of a rivet 6, and it is equipped with a buckle 7 at one of its ends. When the brace 1 is in use, each strap 4 or 5, connected to a shell 2 or 3, is passed round the other shell before being brought back to its own buckle 7, and this strap 4 or 5 is finally made tight and immobilized.

The connection means between the two shells 2 and 3 also comprise, at the base of the brace 1, a stirrup 8 equipped with two symmetrical tabs 9 which are directed upwards. Each tab 9 is engaged through slots 10 formed towards the base of a shell 2 or 3, and it is fixed in a lateral position against a short self-adhesive tape 11 bonded on the outer face of the same shell 2 and 3.

Provided on the inner side of each shell 2 or 3, there is an inflatable bag 12 or 13, respectively. Each inflatable bag 12 or 13, also shown in FIGS. 3 to 5, has a contour which corresponds approximately to that of a shell 2 or 3. This bag 12 or 13 intended to be filled with compressed air is the result of two opposite walls 14 and 15 made of flexible synthetic material which are welded to one another about their perimeter 16. The outer wall 14 of each inflatable bag 12 or 13 is fixed permanently, by adhesive bonding, against the inner face of the corresponding shell 2 or 3.

The two inflatable bags 12 and 13 are each continued in their upper part by flexible inflation conduits 17 and 18, respectively, which converge in a V-shaped configuration towards a common inflation valve 19, as is shown more particularly in FIG. 3 (in which the second, symmetrical bag is not shown) and FIG. 4. It will be noted that the walls of the flexible inflation conduits 17 and 18 are formed directly by continuations of the two opposite walls 14 and 15 of the two inflatable bags 12 and 13. The inflation valve 19 is itself continued by a tongue 20 forming a loop which can be passed through by one of the straps for tightening the brace 1, in particular the lower strap 5 (see also FIG. 2), which guarantees that it is held in place.

In its upper half, each inflatable bag 12 or 13 is provided with a series of weld seams 21 which are parallel and equidistant and extend in a substantially vertical direction and form connections between the two walls 14 and 15 of this inflatable bag 12 or 13, thereby locally limiting the spacing between the walls 14 and 15, even in the case of inflation of the bag 12 or 13 with a high air pressure. FIG. 3 shows the general arrangement of the weld seams 21, while FIGS. 4 and 5 show how the two walls 14 and 15 are maintained at a short distance from one another as a result of this.

Finally, as is illustrated in FIGS. 1, 3 and 5, each inflatable bag 12 or 13 comprises, in its lower half, a small auxiliary bag 22, which is the result of a flexible wall welded on the inner side of said inflatable bag 12 or 13. The auxiliary bag 22, open at its upper part, is intended to receive at least one removable accessory, of corresponding contour, which can be a small comfort cushion 23 of foam and/or a small cushion filled with gel 24, in particular silicone gel.

During the use of the brace 1 described above, and as illustrated in particular in FIG. 2, the two shells 2 and 3 equipped with their respective inflatable bags 12 or 13 are applied on either side of the ankle, while the stirrup 8 (adjusted beforehand) passes under the heel. The two straps 4 and 5 surround the top of the ankle and the bottom of the leg, the inverse or “crossed” assembly of these two straps 4 and 5 ensuring optimal tightening.

The two bags 12 and 13 are inflated simultaneously, via their common inflation valve 19, with the aid of a suitable inflation accessory, in particular an inflation “bulb” (not shown). The general inflation pressure is thus balanced between the two bags 12 and 13, and this pressure is adjustable.

Moreover, by virtue of the connections formed by the weld seams 21 in the upper half of each inflatable bag 12 or 13, the compression obtained decreases towards the top, so that the bearing pressure can be very high at the level of the ankle itself, and much less where the calf starts.

If necessary, still with the aid of the valve 19, the bags 12 and 13 can be partially deflated at any time by the user, without the need to remove the brace 1. This procedure can in particular be performed during the day in the event of increased swelling of the ankle immobilized by the brace.

As the inflation valve 19 is single and common to the two bags 12 and 13, and is situated at the front of the brace, the inflation and deflation of these bags 12 and 13 become particularly rapid and easy procedures.

The positioning of the cushions 23 and/or 24 in the auxiliary bags 22 is optional. It makes it possible to improve comfort in the area of the malleoli and/or to reduce bruising, especially when using a cushion filled with gel 24.

The scope of the invention as defined in the attached claims would not be departed from, irrespective of in particular:
the details of the forms of the various components of the brace;
the materials constituting the components of this brace;
the position of the inflation means which could alternatively be situated at the back of the brace (and not at the front), provided that these inflation means are common to both inflatable bags;
the shape and arrangement of the points or lines of connection of the two walls of each bag, in its upper half;
the accessories which may be fitted on the brace;
the uses of this brace, which can be for various medical indications and therapeutic functions.

1. Inflatable brace for stabilizing the ankle, comprising two lateral shells with which are associated, respectively, two bags which are inflatable with air and which are intended to bear, respectively, on the right and left sides of an ankle, said bags being provided with means for their inflation, and means of the strap type being provided to connect the two shells and permit tightening of the brace around an ankle, characterized in that the means of inflation of the two inflatable bags, respectively right and left, are common to these two bags.

2. Ankle brace according to claim 1, characterized in that the common means of inflation comprise two flexible inflation conduits which start, respectively, from the upper parts of the two bags and which converge, in a V-shaped configuration, towards a common inflation valve situated between the two bags.

3. Ankle brace according to claim 2, characterized in that the two flexible conduits and the inflation valve are situated in the front upper part of the brace.

4. Ankle brace according to claim 2, characterized in that the walls of the flexible inflation conduits are formed directly by continuations of the two opposite walls of the two inflatable bags.

5. Ankle brace according to claim 2, characterized in that the inflation valve is continued by a tongue forming a loop which can be passed through by one of the straps for tightening the brace.

6. Ankle brace according to claim 1, characterized in that each of the two inflatable bags has, at least in its upper half, a zone comprising points and/or lines of connection between its two opposite walls, in such a way as to limit, in this zone, the degree of inflation of each bag.

7. Ankle brace according to claim 6, characterized in that said zone is provided with rectilinear and substantially parallel weld seams which connect the two opposite walls of the bag.

8. Ankle brace according to claim 1, characterized in that each inflatable bag comprises, in its lower half and on the inner side, a small auxiliary bag which is open in its upper part and is intended for the introduction of removable cushions, in particular of foam or gel.
